(19) United States
(12) Patent Application Publication
(10) Pub. No.: US 2009/0228096 A1
(43) Pub. Date: Sep. 10, 2009

(54) FIXATION OF ENDOVASCULAR GRAFTS OR STENT-GRAFTS

(76) Inventors: Edward G. Shifrin, Raanana (IL); Gennady S. Nickelshpur, Haifa, IL (US); Arkady S. Nickelshpur, Herzeliya, IL (US); Wesley S. Moore, Los Angeles, CA (US)

Correspondence Address: OSTROLENK FABER GERB & SOFFEN, 1180 AVENUE OF THE AMERICAS, NEW YORK, NY, 100368403, US

(21) Appl. No.: 12/094,083

(22) PCT Filed: Nov. 17, 2005
(86) PCT No.: PCT/IL05/01217

§ 371 Date: Dec. 9, 2008

(30) Foreign Application Priority Data
Jan. 9, 2005 (IL) 166206

(51) Int. Cl. A61F 2/06

(52) U.S. Cl. 623/1.36; 623/1.23

(57) ABSTRACT

The present invention relates to medical techniques, more particularly, to methods and apparatus for delivery of endovasular devices, such as grafts or stent-grafts, and their oversew fixation to the walls of blood vessels in direction from inside these vessels to their outer surface. There is proposed an apparatus for delivery and oversew fixation of grafts or stent-grafts. the apparatus comprises a tubular body with an expandable working head at the free end having eight cartridges, each of them being provided by one basic and at least one standby fastener means, substantially U-shaped staples, a control mechanism, as well as first and second connecting means. The apparatus is also provided with means for positioning inside a blood vessel. Besides, there is proposed a method for delivery and fixation of a graft or stent-graft to the wall of a blood vessel, substantially the aorta, from inside the latter.

### BACKGROUND OF THE INVENTION

1. Field of the Invention

The present invention relates to medical techniques, in particular, to apparatus and methods used in less or minimally invasive vascular surgery using grafts for eliminating occlusion of blood vessels or stent-grafts for preventing rupture of abdominal aortic aneurysm, as well as to methods for their delivery, location and fixation. More particularly, the present invention relates to methods and apparatus for delivery of intravascular devices, such as grafts or stent-grafts, and securing them to the walls of blood vessels in direction from inside these vessels to their outer surface.

2. Description of Related Art

Occlusion of great blood vessels results from the formation of calcic and/or adipose deposits on their inner surface, or from thrombogenesis and causes deterioration of blood supply to implant organs, such as the heart and liver, which leads to such dangerous situations for the patient, as infarction or insult.

To prevent occlusion of great blood vessels, there are widely used different methods of imaging of main blood vessels. During angiography, inside the blood vessel, there is introduced a guide, and over the latter a catheter with a balloon at the free end. When deposits of essential volume or a thrombus is detected inside the blood vessel, the balloon is inflated by pressure of fluid, so that the corresponding portion of the vessel expands and opens. In case it turns out to be insufficient, and the lumen in the blood vessel remains narrow, there is inserted inside the corresponding portion of this blood vessel a guide with a balloon, carrying on its outer surface a stent in compressed state. When fluid is delivered inside the balloon under pressure, the stent deploys, separates from the balloon and takes the desired position inside the blood vessel. All these manipulations are carried out under X-ray control. Fixation of the stent on the walls of a blood vessel is performed via elastic forces of material the stent is made of, the stent is usually shaped as a spring or has elastic members bearing up against the blood vessel wall or hooking thereon.

When the location of a stent for elimination of narrowing or occlusion of a blood vessel is insufficient, a surgical operation is performed for suturing a natural or synthetic graft.

An aortic aneurysm (or its rupture) is a most common form of arterial aneurysms. It is a very common type of deteriorating disease affecting the ability of a lumen to conduct fluids and may be life threatening. The aneurysm is a ballooning of the wall of an artery or aorta resulting from the weakening of the arterial wall due to disease or other conditions. Left untreated, the aneurysm will frequently rupture, resulting in blood loss through the rupture—the condition, which often leads to death.

The aorta is the main artery, which supplies blood to the circulatory system. The aorta arises from the left ventricle of the heart, passes upwards and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the perineal area and lower extremities.

It is common for an aortic aneurysm to occur in the abdominal region between the renal arteries and the iliac arteries. This portion of the abdominal aorta is particularly susceptible to weakening, resulting in an aortic aneurysm. Such an aneurysm is often located near or including the iliac arteries. An aortic aneurysm larger than about five centimetres in diameter in this section of the aorta is ominous. Left untreated, the aneurysm may rupture, resulting in rapid, and usually fatal, hemorrhaging. Typically, a surgical procedure is not performed on aneurysms smaller than five centimetres because no statistical benefit exists in performing such procedures.

Aneurysms in the abdominal aorta are associated with a particularly high mortality rate; accordingly, current medical standards call for urgent operative repair. Abdominal surgery, however, results in substantial stress to the body. Although the mortality rate for a ruptured aortic aneurysm is extremely high, there is also noticeable mortality and morbidity associated with open surgical intervention to repair an aortic aneurysm. This intervention involves penetrating the abdominal cavity to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aortic wall at the aneurysm.

Beside synthetic grafts, there are developed and used all over the world particularly to prevent the rupture of the aorta wall intravascular devices of “stent-graft” type. They are inserted and positioned similar to stents. Fixation of a stent-graft to the aortic wall is performed via elastic forces of the material of the stent-graft itself which usually has springy elements bearing up against the blood vessel wall or hooking thereon. When the forces of elastic or springy elements of the stent-graft are insufficient for its fixation in a blood vessel, the stent-graft may be displaced from the assigned position and moved along the aorta under the action of blood flow and peristelsic oscillations of the wall of this blood vessel, and that is quite dangerous for the patient.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, the common repair means is to deploy a graft or a stent-graft within the lumen of the affected aorta/artery in the region of the aneurysm. These methods and devices have been developed to attempt to treat an aortic/arterial aneurysm without the attendant risks of intra-abdominal or open arterial surgical intervention. Among them are inventions disclosed and claimed in Parodi, Juan C. et al., WO 010487A1 for Graft Device for Treating Abdominal Aortic Aneurysm and its patent family, including U.S. Pat. Nos. 5,219,355, 5,522,880, 5,571,171, 5,643,208, 5,683,452, 5,693,087, 6,102,942, EP 461791A1, EP 809980A3, EP 903118A2, EP 903119A3, EP 903120A3 etc..

In other Parodi's patents there are disclosed stent-graft designs having a metal wire frame collapsible to a minimal size sufficient to insert the stent-graft into the artery through a puncture in its wall and expandable inside the aorta to a required size under the action of a radial force, such as a balloon. This frame is covered by a sheath which can contract and expand together with the frame under the action of external forces. Aforesaid stent-grafts are provided with means for mechanical fixation to the walls of the aorta or iliac arteries. Among those means we find balloon cuffs of a special shape at the stent-graft ends, see WO 010487A1, U.S. Pat. No. 5,219,355, various hooks, elements shaped as scales, spirals and similar elements designed for fixation on the wall of the aorta or artery, see U.S. Pat. No. 5,911,733 “Endovascular Expander of a Non-migrant Positioning”, EP 948945 A2 “Endovascular Prosthesis with Fixation Means”.

Migration of grafts and/or stent-grafts from determined positions concerning walls of aorta or major arteries is still a very actual problem which increases the risk of surgical procedure with introduction of these prostheses. In different countries there are developed various devices and methods for the fixation of prostheses. As fastener means there are suggested helical fasteners, see U.S. Pat. No. 6,562,051, U.S. Pat. Appl. 2003/0135226 A1 or spiral holders, see EP 1300121 A2, U.S. Pat. Appl. 2003/0093146 A1, or endoluminal anchors, see U.S. Pat. Nos. 6,746,472, 6,231,561 or 6,328,727, and as a means for their delivery and location devices like “Endovascular aneurysm repair system”, see WO 03/045283 A1.

There are further known numerous inventions the authors whereof suggest fastener means made of a shape memory alloy. Among these are inventions by Howard Tanner, Arnold Miller etc., see WO 01/58364 A1, U.S. Pat. Nos. 5,944,750, 5,972,023, WO 02/17797 A1, WO 02/17796 A1, WO 02/19923 A1 and U.S. Pat. Appl. 2002/0029048 A1 and 2003/0105473 A1.

And, at last, fastener means are known produced by “Anson Medical Ltd”, see Britain Pat. GB 2359024A “Fixator for arteries”, WO 01/58363 A1 and U.S. Pat. Appl. 2003/0033006 A1 “Device for the repair of arteries”. This device for retaining a graft on an artery comprises an abutment portion for abutting the graft wall, and two elongated members extending therefrom. The elongate members are resiliently biased into a retaining configuration and moveable into an axial configuration to enable the fixator to be conveyed along an artery. The device comprises at least two wires fabricated from a material which has a spring of thermal shape memory alloy such as nickel/titanium shape memory alloy such that their unconstrained shape is a curved “Y” or “gull-wing” shape. The wires can be constrained to be parallel, each wire having two ends, the wires being joined together at or near to one end by welding, braising or similar means. In other embodiments the device is made from one wire and has a “gull-wing” shape.

While performing surgical operations using stents, grafts or stent-grafts, physicians and engineers face the problem of delivering implanted devices through a comparatively small opening in the artery (up to 6-8 mm in diameter), then through the artery inside the operated blood vessel, such as the aorta, and succeeding deployment of the implanted device in the lumen of this blood vessel. The lumen size in the aortic neck area is at least 20 mm. Hence, the delivery system together with the implanted device mounted thereon must have, firstly, a comparatively small outer diameter to pass through this punch in the artery wall, and, secondly, be capable of essentially enlarging its cross section at the spot where the implanted device should be located. For this purpose the delivery system is usually provided with an inflatable hydraulic balloon carrying on its surface the delivered device—stent, graft or stent-graft. At the same time, there are known entirely mechanical delivery systems, wherein instead of a balloon mechanical deployable means are used resembling Chinese lanterns, such as “Mechanical stent and graft delivery system” described by Houser, Russell et al., see U.S. Pat. No. 6,217,585 or “Apparatus and method for dilating a lumen and for inserting an intraluminal graft”, see Hogendijk, Michael et al., U.S. Pat. No. 5,713,907.

Similar to the latter is the “Cardiovascular mechanically expanding catheter” by Bar-Cohen, Yaniv, see U.S. Pat. No. 5,855,565. The catheter has a mechanically expanding mechanism on the distal end thereof for dilating vessels such as arteries and other endoluminal structures for the deployment of intraluminal stents. A permeable, mechanical dilating mechanism includes a pair of expanders which provide a radial force against walls of the vessel in response to a pulling or contracting longitudinal force on the dilating mechanism, for example, by means of a cable. A set of adjacent quadrates are preferably provided for promoting uniformity of pressure by the expanders.

Among mechanical delivery systems, closest to the claimed invention are methods and devices developed by Marin, Michael and Marin, Ralph, see U.S. Pat. Nos. 5,443,477, 5,507,769, 5,591,196, 5,618,300, 5,695,517, 6,039,749, 6,168,610, 6,575,994, as well as WO 95/21593, WO 96/11648 and EP 1290989. By the disclosed method, a stent-graft complex is advanced through each branch of the patient's femoral and iliac systems. The stent-graft complex includes a segment of prosthetic graft material attached at each end to a respective stent. The proximal part of each stent-graft complex is positioned relative to one another in a common region of normal aortic tissue on one side of the aneurysm and then deployed. The distal part of stents is deployed in the iliac arteries. Additional steps can be taken to ensure that the internal iliac artery is not blocked when the distal stents are deployed.

A common drawback of all above mentioned methods and devices is that they are chiefly intended for delivering implanted devices inside operated blood vessels. The delivered implanted devices are provided at their ends with fixation means—stents which are not incorporated in the structure of delivery devices. Besides, in our opinion, the fixation of implanted devices must be performed substantially by elastic force of these stents. We think it is obviously insufficient and cannot prevent the displacement of implanted devices due to blood flow and peristelsic oscillations of blood vessel walls. Besides, the length of aorta neck does not always allow to locate a stent at this place which makes conjectural the application of the aforesaid methods and devices.

We contemplate that delivery systems must have, apart from means for delivery and deployment of implanted devices, special means for fixation of implanted devices to blood vessel walls, such as the aorta, to provide their secure fixation in required places and prevent thereby displacement of implanted devices via aforesaid forces.

There are known devices used during surgical operations for suturing tissues via fixing means, such as clips, see, for example, suturing instrument and clip by Schulken, Heinrich et al., U.S. Pat. Nos. 5,499,990, 5,522,823 or surgical staple for tissue by Richard, Thierry, see France Pat. 2746292.

These British company “Anson Medical Ltd” and its specialists, Anson, Antony et al., see EP 0915678 B1 and WO 00/07506, disclose a device for retaining a graft on an artery, comprising a first part for contacting with the graft and a second part for contacting with the artery when the device is pierced radially through the graft and the arterial wall. This first and second parts are connected by a resilient member, which biases the first and second parts towards each other into a retaining configuration such that in use the artery and the graft are retained together between the first and second parts of the device. The device for graft fixation is made of a shape memory alloy, and includes in transport position two lengths of wire arranged in parallel relationship to one another and fastened together at the middle. To deliver this device to the location spot there is used a delivery system including an expandable member for the graft deployment in its fixation area and for pressing the graft edge against the aorta wall, as well as means for storage and delivery of the described devices for graft fixation to the spot of their location.

Suyker, Wilhelmus et al., in U.S. Pat. No. 6,485,496 and U.S. Pat. Appl. 2003/0055441 disclose a system for making anastomoses between hollow structures by mechanical means, which is provided with a device in the shape of an annular or tubular element comprising circumferentially provided means, such as pin-shaped elements, for joining the abutting walls of the hollow structures together. There is also disclosed an applicator for moving this annular or tubular element in position and activating the joining means thereof, so as to make the anastomosis.

3. The Prior Art

Parodi, Juan, in U.S. Pat. No. 6,336,933, EP 1308131 A1 and U.S. Pat. Appl. 2003/0023248 discloses systems and methods for applying a suture within a blood vessel lumen. The systems and methods advance within a blood vessel a catheter tube having a distal region that carries a suture applicator. The systems and methods operate the suture applicator from a location external to the blood vessel lumen to apply a suture, having a spiral shape, to the interior wall of a blood vessel lumen. In U.S. Pat. No. 6,592,593 and WO 00/16101, Parodi, Juan et al., disclose an applicator including a tubular body that is configured for positioning within a vessel, an expandable portion disposed adjacent to the distal end of the tubular body and deployed to support a prosthetic in contact with the inner surface of a vessel, and a drive assembly for advancing a fastener into the prosthetic. A delivery tube may be included that is disposed for movement within the tubular body, and which defines a channel for movement of the drive assembly therein. The delivery tube may include an applicator head. The applicator head may include an injection mount that is disposed for movement relative to a prosthetic.

Closest to the present invention is “Second generation coil fastener applier with memory ring” by Aranyi, Ernest et al., see U.S. Pat. Appl. 2002/0177862 and WO 00/64357. There is an endovascular fastener applier for fastening a vascular graft to a vessel with at least one fastener. The applicator generally includes a tubular body configured for positioning within the vessel, and an expandable portion adjacent to the tubular body. A fastener applying head is movably mounted within the expandable portion. The applicator further includes a handle assembly mounted on a proximal end of the tubular body. The handle assembly generally includes a control for releasing the expandable portion, pivoting, rotating the fastener applying head and for driving the fastener out of a fastener driving head. Preferably, the applicator also includes a storage chamber extending from a distal end of the expandable portion, and containing a plurality of fasteners.

The known methods and systems for delivery or fixation of grafts and stent-grafts have, as a rule, different functions, i.e. serve solely either for dilivery and deployment of grafts and stent-grafts or for their fixation to blood vessel walls. Those few methods and systems that combine these functions, see, for example, Marin, Michael and Marin, Ralph, U.S. Pat. Nos. 5,443,477, 5,507,769, 5,591,196, 5,618,300, 5,695,517, 6,039,749, 6,168,610, 6,575,994, WO 95/21593, WO 96/11648 and EP 1290989, use for prostheses fixation stents or similar devices connected to these prostheses, and these devices do not provide secure fixation of implanted prostheses to the walls of corresponding blood vessels and the aorta.

An object of the present invention is to develop a method and apparatus providing simultaneous delivery and secure oversew fixation of implanted endovascular prostheses, particularly, grafts or stent-grafts, in corresponding blood vessels.

Another object of the present invention is to develop a system of apparatus assembled from a kit of standardized parts ( similar to a construction kit for children) which allows to assemble fast an apparatus adapted to the anthropometric conditions of a specific patient and intended for performing a surgical operation to deliver a graft or stent-graft of a specific type and size and its oversew fixation to the walls of a blood vessel.

A third object of the present invention is to develop a new method of grafts or stent-grafts oversew fixation to the walls of blood vessels.

### SUMMARY OF THE INVENTION

The subject matters of the present invention are an apparatus and method for delivery and oversew fixation to the walls of blood vessels, substantially the aorta, of endovascular devices, substantially grafts or stent-grafts. The proposed apparatus comprises a tubular body, expandable working head with cartridges adapted for location of fastener means, as well as a control mechanism.

The tubular body of this apparatus is configured for positioning inside a blood vessel. This tubular body is rigid in longitudinal direction and flexible in lateral direction.

The working head is disposed near the free end of said tubular body and is expandable for retaining a delivered endovascular device in contact with the inner surface of a blood vessel at the moment of their mutual fixation. The working head is provided at least with eight cartridges for location of fastener means, the cartridges being incorporated in the structure of this working head. The cartridges are provided with means for retaining therein fastener means, for imparting thereto progressive motion, for shaping fastener means during their extension from these cartridges, as well as for storage and delivery of standby fastener means.

The control mechanism of this apparatus is provided at least with one first control means and at least with one second control means. The control mechanism is disposed at the end of said tubular body opposite to the working head, and is associated with the expandable working head via this tubular body containing a retaining tube, rigid in longitudinal direction and flexible in lateral direction. The first control means serves for control of the expansion and contraction of this expandable working head, and the second control means serves for control of the extension of fastener means from each of at least eight cartridges, as well as for control of recharging of each of these cartridges at least with one standby fastener means.

The working head contains a pressure bush, a first and a second bearing bushes arranged in succession one after another beginning from the free end of this head and in alignment with the, tubular body, as well as an axial tube rigidly connected with the first bearing bush and passing via a through axial hole in the second bearing bush.

The expandable working head is provided with eight cartridges. The cartridges of this working head are pivotally mounted by one their ends on the first bearing bush, and by their other ends pivotally connected with bearing levers pivotally mounted on the second bearing bush. The expandable working head is further provided with means for securing a delivered graft or stent-graft disposed on bearing levers near their pivotal connections with corresponding cartridges.

Each cartridge of the working head contains at least one basic fastener means, substantially a U-shaped staple arranged in this cartridge substantially along its longitudinal axis, with free pointed ends facing the means for shaping located in each cartridge near the spot of its pivotal connection with a corresponding bearing lever. The U-shaped staple is disposed in the cartridge so as to progressively extend therefrom and be shaped during this extension. Further, each cartridge contains a means for retaining therein at least one fastener means, substantially U-shaped staple, this means for retaining being arranged along the cartridge and containing a sliding lid.

At last, each cartridge includes at least one means for imparting progressive motion to the fastener means, substantially U-shaped staple. This means contains a flexible pusher, rigid in longitudinal direction and flexible in lateral direction. All the flexible pushers are associated by one their end with the pressure bush of said expandable working head and disposed under sliding lids of corresponding cartridges to reciprocate in the clearance between the bottom of a corresponding cartridge and its sliding lid.

Each cartridge has a bottom and a sliding lid and contains a means for storing at least one standby fastener means, substantially a standby U-shaped staple. This means for storing contains a slot in the cartridge bottom wherein at least one spring-loaded standby U-shaped staple is located disposed in this slot substantially along the longitudinal axis of this cartridge, with free ends facing the means for shaping. At least one standby U-shaped staple is disposed in the slot of this cartridge bottom substantially under its flexible pusher and is capable of extension from this slot by action of the spring and at partial removal of the flexible pusher from the clearance between this cartridge bottom and its sliding lid.

In another cartridge embodment the slot in its bottom is a through one, and the standby U-shaped staple is located in this slot immediately under the flexible pusher and spring-loaded on the opposite side.

Each cartridge of the endovascular apparatus contains at least one basic and at least one standby fastener means, substantially U-shaped staples. All these staples are fabricated from one of the materials of a group including stainless steel, titanium and shape memory alloys.

All eight cartridges of the working head are evenly arranged about the longitudinal axis of this head and pivotally connected by one their ends with its first bearing bush, and by its other ends pivotally connected with bearing levers which are, in their turn, mounted on the second bearing bush. All the cartridges together with their sliding lids, flexible pushers and bearing levers are identical and interchangeable.

The apparatus is further provided with means for axial and radial positioning in a given point of the operated blood vessel. The means for axial positioning contains a measuring scale on the surface of said tubular body. The means for radial precision positioning of the working head contains markers—X-ray contrast marks evenly applied on the outer surface of cartridges near the spots of their connection with bearing levers and serving for orientation of this expandable working head by its operation angle.

In the proposed apparatus the first bearing bush is connected via the first connecting means with said first control means of the control mechanism. The pressure bush is operatively connected via the second connecting means with the second control means of this control mechanism. The second bearing bush is connected with the body of this control mechanism via a retaining tube serving as a tubular body. The first connecting means and second connecting means are enclosed in said retaining tube concentrically with one another and with this retaining tube. The first connecting means contains an axial tube, and the second connecting means may be substantially a tie.

The control mechanism contains a hollow body with a first and second end rigidly connected by its first end with said working head via the retaining tube. Further, this control mechanism has a slider enclosed in this body to reciprocate relative to the latter. The slider is provided with a retaining handle rigidly secured thereto, operatively associated with a pivoting head located at the second end of said hollow body and forms together with this pivoting head the first control means of said control mechanism. The control mechanism also contains a pressure handle serving as said second control means of this control mechanism. This pressure handle is rotatably secured to the retaining handle and has a short free end enclosed in the slider. The slider is rigidly connected with the axial tube serving as said first connecting means, and the short free end of said pressure handle is operatively associated with the tie serving as said second connecting means. This pressure handle is provided with a swing lock pivotally mounted on the slider, as well as with a flat return spring secured by one its end to this pressure handle, and by its second end to the retaining handle.

Each cartridge of the proposed apparatus has a body with a bottom provided with a recess for location of at least one basic fastener means, substantially U-shaped staple, and a through slot for location of at least one standby fastener means, substantially standby spring-loaded U-shaped staple. Further, the body of each cartridge has slots for mounting a sliding lid, as well as a longitudinal duct for location of at least one flexible pusher. The basic U-shaped staple is disposed in its recess in frictional contact with the sliding lid to prevent it from falling out of this recess. The standby spring-loaded U-shaped staple is disposed in its through slot in frictional contact with the flexible pusher to prevent it from extension from this through slot before partial removal of this flexible pusher.

Each cartridge of the working head has a body with a bottom which has a recess for location of at least one basic fastener means, substantially U-shaped staple. The recess in this cartridge body is symmetric about the longitudinal axis of said cartridge and parallel with its outer surface. The cartridge is further provided with a means for shaping the U-shaped staple during its extension from the recess. This means contains a curvilinear guiding surface connecting the bottom of said recess for the U-shaped staple with the cartridge outer surface. This curvilinear guiding surface has shaping grooves diverging at an acute angle to one another and to the longitudinal axis of this cartridge.

Each U-shaped staple has pointed free ends and is located in the body recess of a corresponding cartridge in such a way that its pointed ends face corresponding shaping grooves on the curvilinear guiding surface of this cartridge. This provides helical oppositely directed curling of pointed ends of a corresponding U-shaped staple during its movement over the body recess and shaping grooves of the curvilinear guiding surface of this cartridge.

Each cartridge of the working head is shaped substantially as a polyhedral prism with two skewed side faces inclined at an acute angle to one another. The vertex of this acute angle is located on the longitudinal axis of said expandable working head. All cartridges are provided with means for connection with this expandable working head. These means substantially contain arms with holes projecting from both bases of a corresponding polyhedral prism and being integral with this polyhedral prism.

All bearing levers of the expandable working head are provided with means for securing a delivered graft or stent-graft shaped substantially as radial tongues which are disposed at one of the ends of these bearing levers, near pivotal connections with the cartridges.

Another subject matter of the present invention is a method for delivery and oversew fixation of a graft or stent-graft to the wall of a blood vessel from inside the latter, which comprises several successive steps.

At the first step the graft or stent-graft is prepared for delivery, mounted and secured on the surface of said expandable working head of the apparatus for delivery and fixation, as well as crimped to an assigned outer diameter.

At the second step the endovascular apparatus for delivery is brought into operative position, inserted into a corresponding blood vessel, the expandable working head is brought to the securing area, and there is performed precision axial and radial positioning of the appratus in a given aorta point via a measuring scale on the surface of said tubular body and via markers—X-ray contrast marks. Then the working head is deployed in such a way that the delivered graft or stent-graft gets in contact with the inner surface of said blood vessel at the moment of their mutual fixation, and the cartridges of this expandable working head are located near corresponding suturing points.

Thereupon U-shaped staples are set in motion, which is accomplished by action of flexible pushers activated, in their turn, via the pressure bush, second connecting means and second control means which are associated with these pushers. As a result, pointed ends of each U-shaped staple enter the forming grooves of said curvilinear guiding surface of a corresponding cartridge, are curled therein, diverging in opposite direction from one another, pierce the graft or stent-graft wall and surrounding blood vessel wall and return again over a spiral to the curvilinear guiding surface, repeating this rotation if permitted by the given length of staples. As a result, the graft or stent-graft wall gets sutured by wire spirals formed from U-shaped staples to a corresponding portion of this blood vessel, and the U-shaped staples themselves emerge from recesses of corresponding cartridges and are released from these cartridges, forming an oversew suture at the end of a corresponding graft or stent-graft.

Then the cartridges are recharged with standby U-shaped staples, which is accomplished via partial removal of flexible pushers activated, in their turn, via the pressure bush, second connecting means and second control means which are associated with these pushers. As a result, each spring-loaded standby U-shaped staple extends from the through slot of a corresponding cartridge, and then, at reverse movement of a corresponding flexible pusher, moves due to its action into a recess where the basic U-shaped staple was located before. As a result, the endovascular apparatus is made ready for a repeated prosthesis suturing.

Then, if repeated suturing is necessary, standby U-shaped staples are set in motion. This is accomplished by flexible pushers activated, in their turn, via the pressure bush, second connecting means and second control means much as basic staples were extended before. As a consequence, pointed ends of each standby U-shaped staple enter the shaping grooves of said curvilinear guiding surface of a corresponding cartridge, are curled therein, diverging in opposite sides from one another, pierce the graft or stent-graft wall and surrounding blood vessel wall and return again over a spiral to this curvilinear guiding surface, repeating this rotation if permitted by the given length of staples. As a result, the graft or stent-graft wall gets repeatedly sutured by wire spirals formed from standby U-shaped staples to a corresponding portion of the blood vessel, and standby U-shaped staples themselves entirely extend from the recesses of corresponding cartridges and are released from these cartridges forming an oversew suture at the end of a corresponding graft or stent-graft.

At last, at the final step, the delivered and sutured graft or stent-graft is detached from the apparatus for delivery and fixation, this apparatus is brought in inoperative position and removed from the graft or stent-graft and from the blood vessel.

### BRIEF DESCRIPTION OF THE DRAWINGS

The invention will now be described in conjunction with the following drawings in which like reference numerals designate like elements and wherein:

FIG. 1 shows a general view of the proposed apparatus.

FIG. 2-4 show a longitudinal section of the control mechanism of this apparatus, its tubular body and working head;

FIG. 5-6 show a general view of the 8-cartridge working head of the proposed apparatus in partly deployed position;

FIG. 7 shows the general view of a cartridge with a sliding lid;

FIG. 8 shows the general view of a bearing lever;

FIG. 9 shows a general view of the first bearing bush of the working head;

FIG. 10 shows the general view of a cartridge with a sliding lid and arms for securing this cartridge;

FIG. 11 shows the general view of a cartridge with a through slot in its bottom;

FIG. 12 shows the general view of a cartridge as viewed from the through slot;

FIG. 13, 14 show views of a cartridge with diagrams of location of a basic and standby U-shaped staples;

FIG. 15 shows an operation diagram of the proposed apparatus.

### DETAILED DESCRIPTION OF PREFERRED EMBODIMENTS

The preferred embodiments of the present invention are described below. The inventors of the present subject matter contemplate that the embodiments described herein are capable of use in the repair of other vessels and in other procedures. Thus, it is intended that the present invention cover the modifications and variations of the invention, provided they come within the scope of the appended claims and their equivalents.

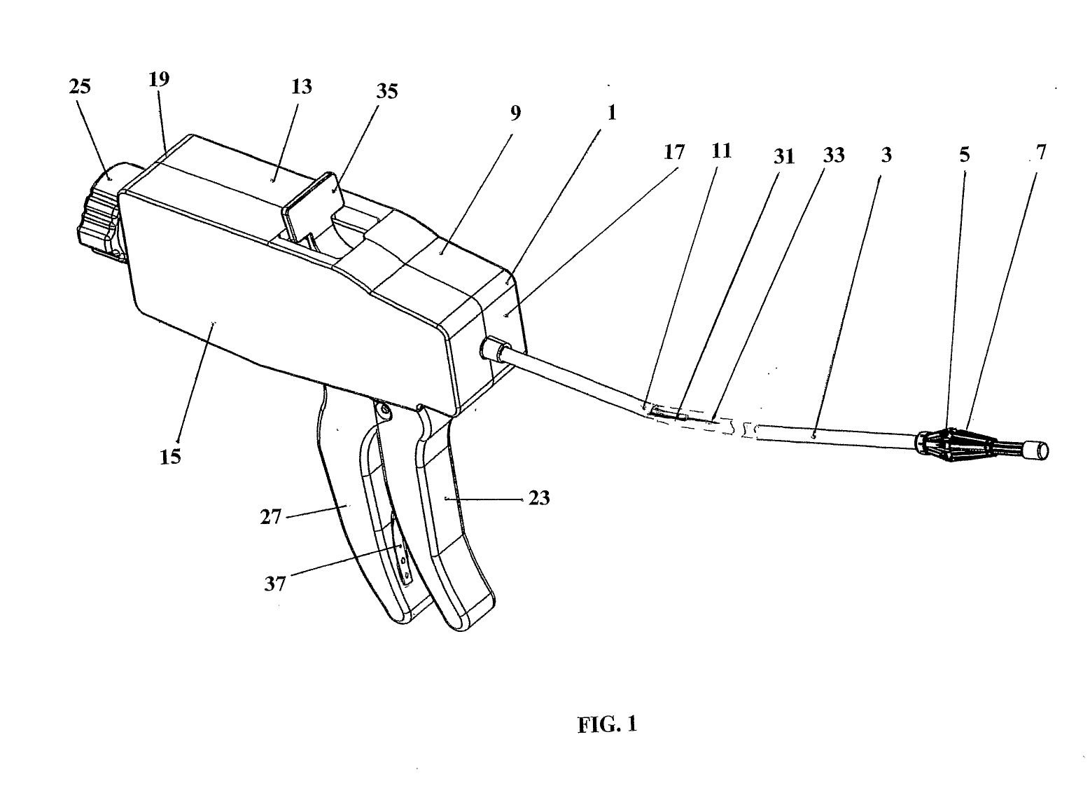

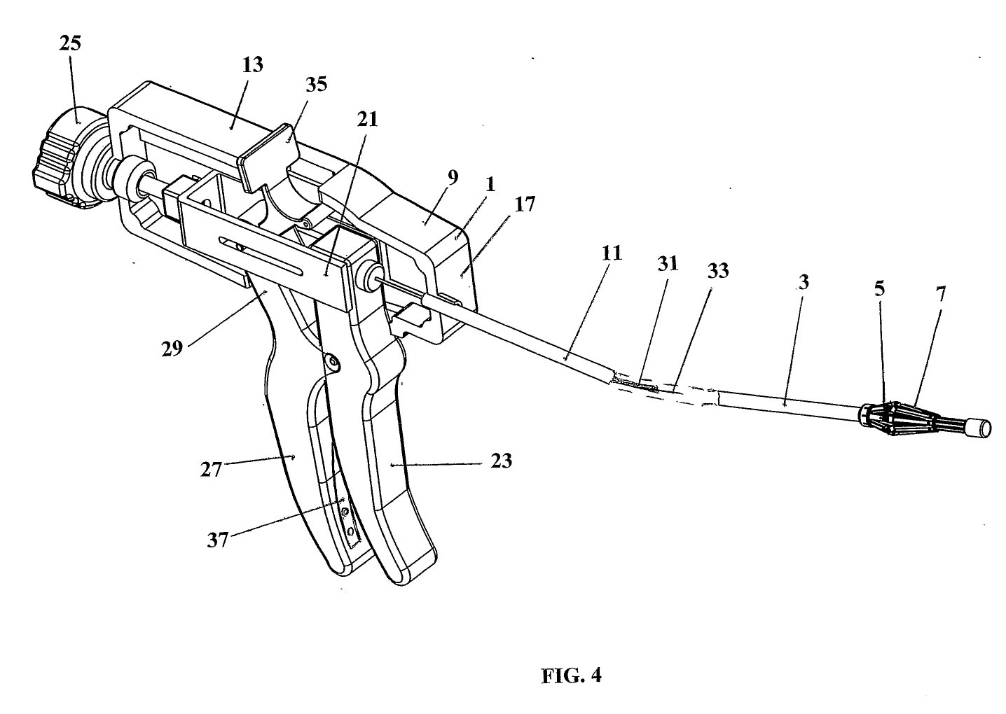

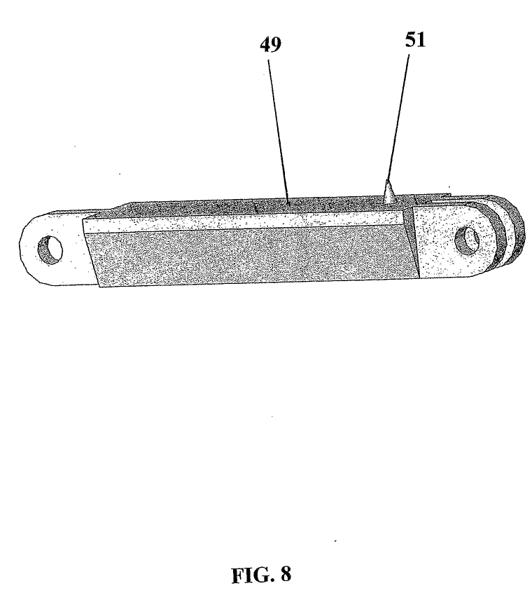

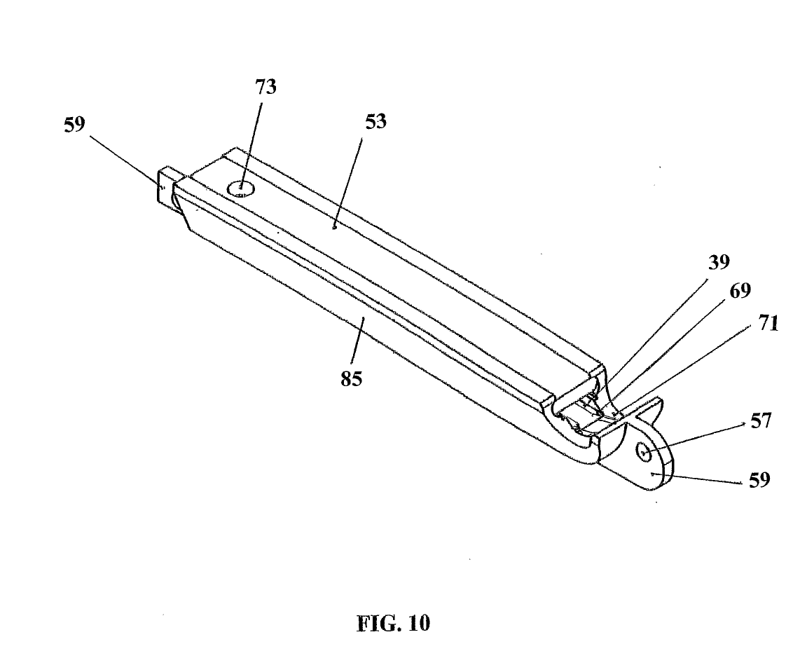

The most preferred embodiment of an apparatus, according to the present invention, are shown in drawing FIGS. 1-15.

The subject matter of the present invention is an apparatus 1 (FIG. 1-4) for delivery and oversew fixation to the walls of blood vessels, substantially the aorta, of intravascular devices, such as grafts or stent-grafts. This apparatus 1 comprises a tubular body 3, expandable working head 5 with cartridges 7 for location of fastener means, as well as a control mechanism 9.

Tubular body 3 of apparatus 1 is configured for positioning inside a blood vessel. This tubular body 3 contains a retaining tube 11, rigid in longitudinal direction and flexible in lateral direction (FIG. 1).

Control mechanism 9 contains a hollow body 13 with a lid 15, as well as with a first and second ends, 17 and 19 respectively (FIG. 1, 2). Hollow body 13 is rigidly connected by its first end 17 with working head 5 via retaining tube 11. Control means 9 is provided at least with one first control means and at least one second control means. The first control means consists of a slider 21 located in body 13 of control mechanism 9 to reciprocate relative to the latter (FIG. 1-4). Slider 21 is provided with a retaining handle 23 rigidly attached thereto, operatively associated with pivoting head 25 located at the second end 19 of body 13 and forms together with this pivoting head 25 the first control means of this control mechanism. Control mechanism 9 also contains a pressure handle 27 serving as the second control means of this control mechanism.

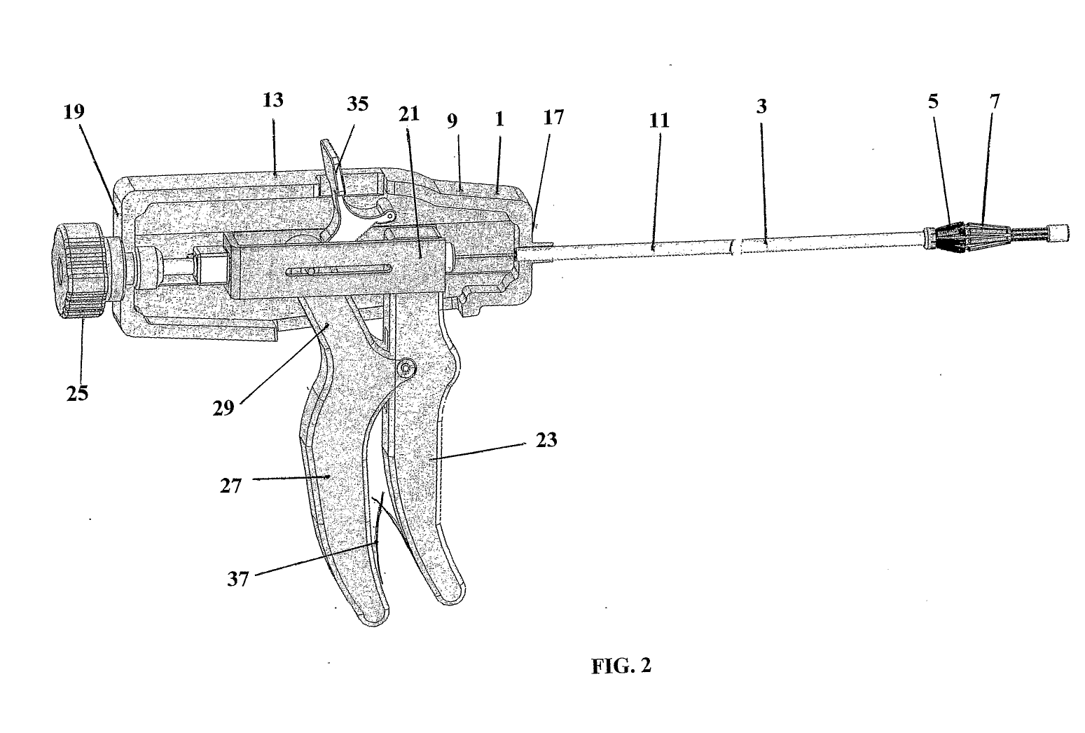

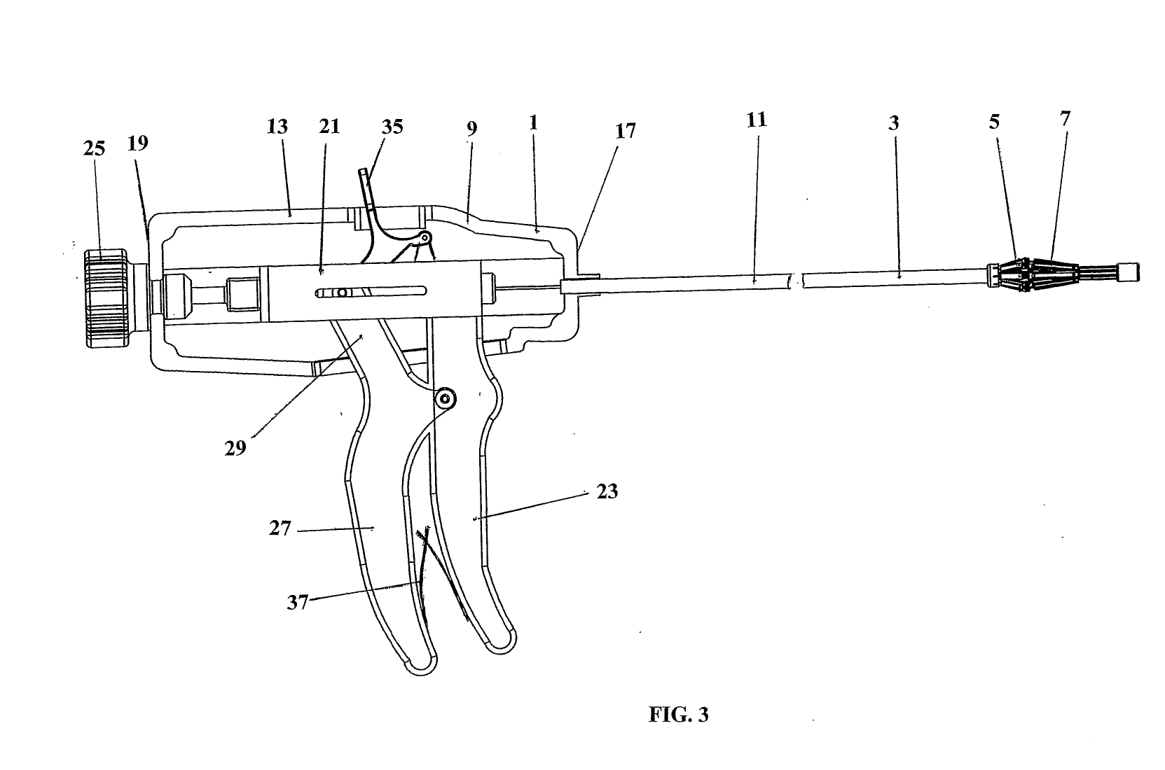

Pressure handle 27 is pivotally mounted on retaining handle 23 and has a short free end 29 enclosed in slider 21. Slider 21 is rigidly connected with axial tube 31 serving as a first connecting means, and short free end 29 of pressure handle 27 is operatively associated with tie 33 serving as a second connecting means (FIG. 1-4). Pressure handle 27 is provided with a swing lock 35 pivotally mounted on slider 21, as well as with flat return spring 37 secured by one its end to this pressure handle 27, and by its other end to retaining handle 23 (FIG. 2, 3, 4). Swing lock 35 in its operative position limits the reverse motion of slider 21 (FIG. 3) in a position wherein no recharging of cartridges 7 with standby U-shaped staples is required, and return spring 37 located between pressure handle 27 and fixed handle 23 (FIG. 3) provides reverse movement of pressure handle 27.

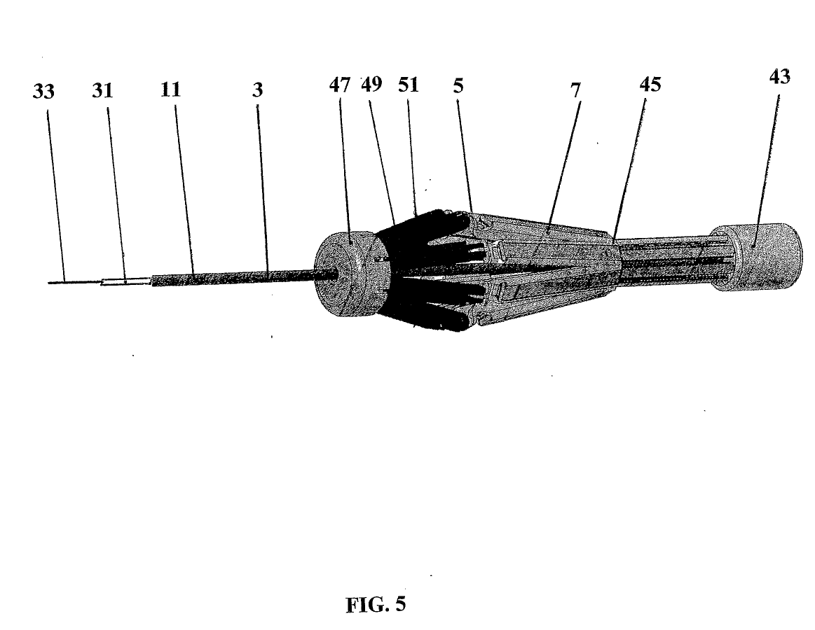

The first control means—slider 21 with retaining handle 23 and pivoting head 25 serves to control the expansion or contraction of expandable working head 5, and the second control means—pressure handle 27 serves to control the extension of fastener means from each of at least eight cartridges 7, as well as to control the recharging of each of these cartridges 7 at least with one standby fastener means (FIG. 1-4). The first connecting means and the second connecting means, i.e. axial tube 31 and tie 33 are enclosed in retaining tube 11 concentrically with one another and with this retaning tube 11 (FIG. 5, 6).

Working head 5 is disposed near the free end of tubular body 3 and is expandable to retain a delivered intravascular device in contact with the inner surface of a blood vessel at the moment of their mutual fixation. Working head 5 (FIG. 5, 6) is provided at least with eight cartridges 7 for location of fastener means, substantially U-shaped staples 39, cartridges 7 being incorporated in the structure of working head 5. Cartridges 7 are provided with means for retaining therein U-shaped staples 39, for imparting thereto progressive motion and for shaping these U-shaped staples 39 during their extension from these cartridges 7, as well as for storage and delivery of standby fastener means—the same U-shaped staples 41.

Working head 5 (FIG. 5, 6) contains a pressure bush 43, first and second bearing bushes, 45 and 47 respectively, arranged in succesion one after another beginning from the free end of this head 5 and in alignment with retaining tube 11 of tubular body 3, as well as an axial tube 31 rigidly connected with first bearing bush 45 and passing via through axial hole in second bearing bush 47.

All eight cartridges 7 of working head 5 are evenly arranged about the longitudinal axis of this head 5 and pivotally connected by one their ends with its first bearing bush 45, and by its other ends pivotally connected with bearing levers 49 which are pivotally mounted on second bearing buh 47 (FIG. 5, 6). Expandable working head 5 is further provided with means for securing a delivered graft or stent-graft disposed at one of the ends of bearing levers 49, near their pivotal connections with corresponding cartridges 7 which are shaped substantially as radial tongues 51.

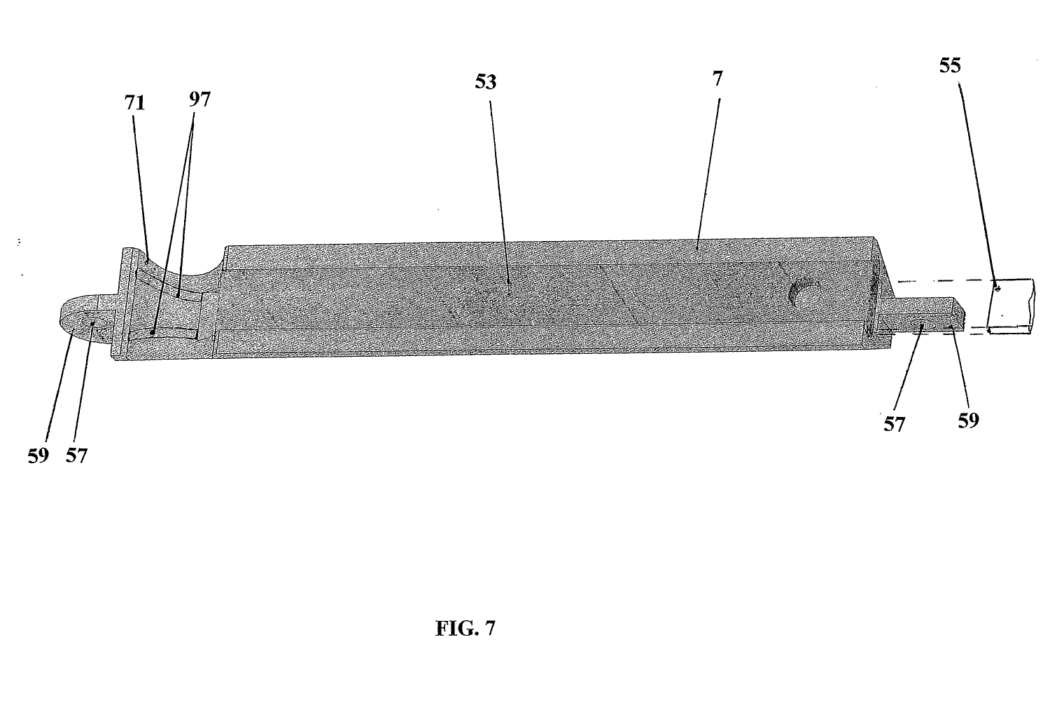

All eight cartridges 7 of working head 5 are pivotally secured to bearing levers 49 and provided with means for retaining therein fastener means, in this case sliding lids 53 (FIG. 7, 8, 10), for imparting progressive motion thereto—flexible pushers 55, as well as with means for shaping fastener means—U-shaped staples 39, said means being shown hereinbelow in more detail, as well as for storage and delivery of standby fastener means—U-shaped staples 41.

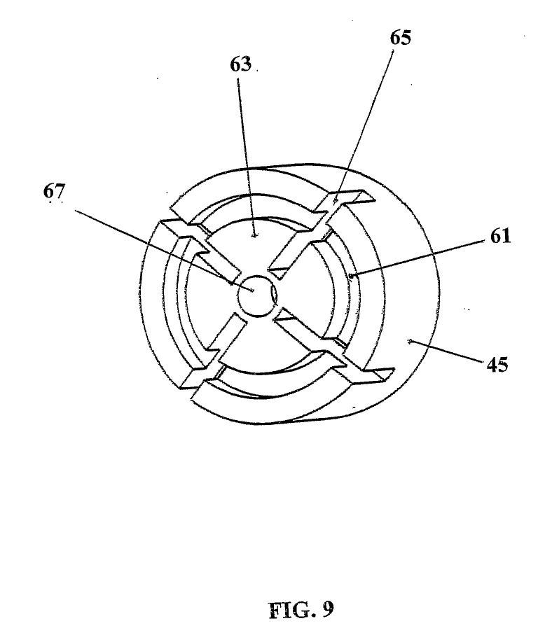

All flexible pushers 55 are associated by one its end with pressure bush 43 of expandable woking head 5 and arranged about first bearing bush 45 (FIG. 5, 6). Expandable working head 5 is provided with elements for pivotal attachment of cartridges 7 (FIG. 7). These may be for instance fitting rings whereon cartridges 7 are mounted by holes 57 of their arms 59. These rings are tightly located in special grooves 61 in end face 63 of first bearing bush 45 (FIG. 9). Arms 59 of cartridges 7 are located in slots 65 specially provided for them on end face 63 of bearing bush 45 and may rotate in these slots 65 about a corresponding fitting ring as about a pivot. Pivotal attachment of bearing levers 49 to second bearing bush 47 (FIG. 7, 8) is much the same. Axial hole 67 in first bearing bush 45 serves for passing tie 33 through this bush 45 (FIG. 9).

Each cartridge 7 of working head 5 contains at least one basic fastener means, substantially U-shaped staple 39 located in this cartridge 7 substantially along its longitudinal axis, with free pointed ends 69 facing the means for shaping—curvilinear guiding surface 71 provided in each cartridge 7 near the point of its pivotal connection with a corresponding bearing lever 49 (FIG. 7, 10). U-shaped staple 39 is disposed in cartridge 7 to progressively extend therefrom and be shaped during this extension. Further, each cartridge 7 contains a means for retaining therein U-shaped staple 39, this means for retaining being located along the cartridge and shaped as a sliding lid 53 with hole 73 for its opening and closing. All flexible pushers 55 associated by one end with pressure bush 43 of expandable working head 5 are located under sliding lids 53 of corresponding cartridges 7 to reciprocate in the clearance between the bottom of a corresponding cartridge 7 and its sliding lid 53.

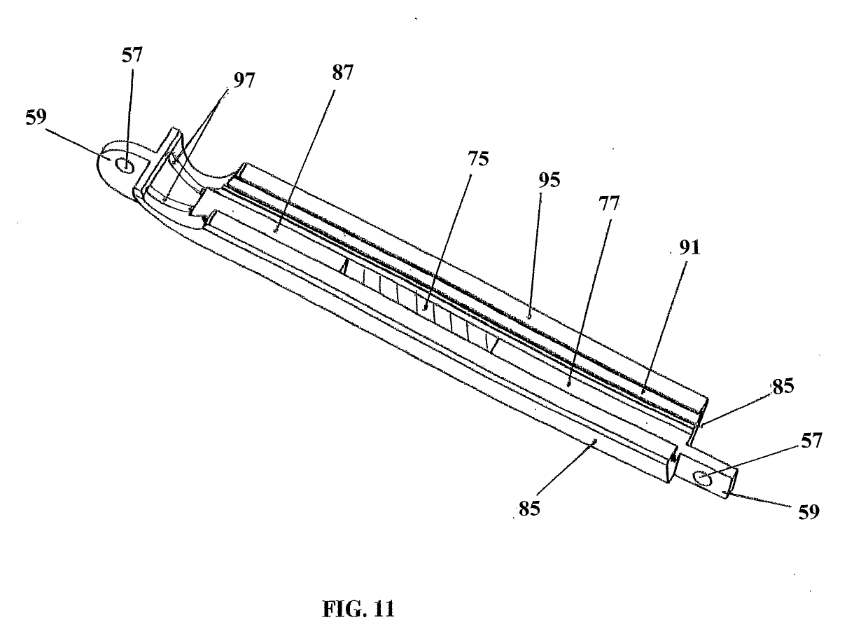

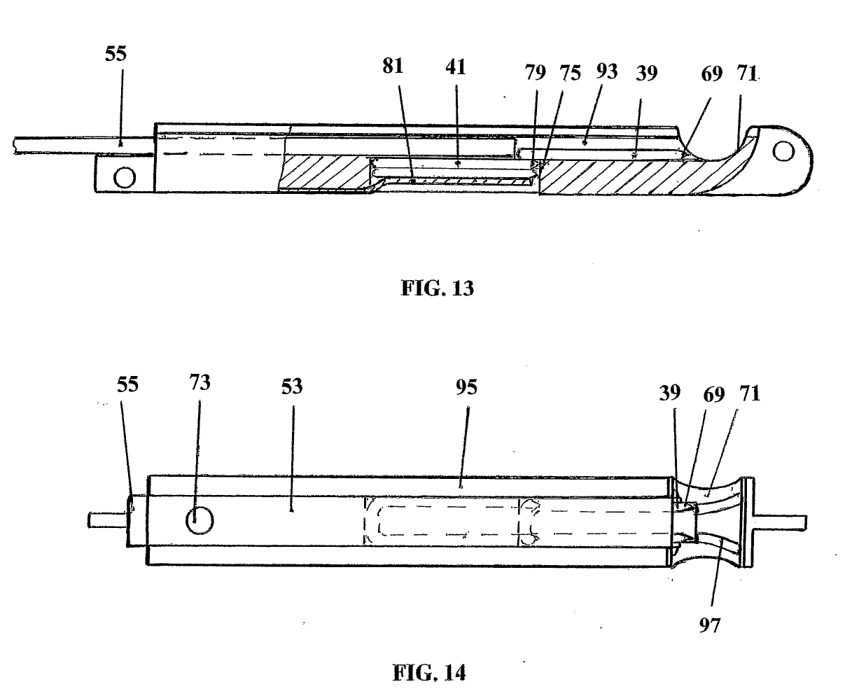

All cartridges 7 contain a means for storing at least one standby U-shaped staple 41 (FIG. 11-14). This means for storing is shaped as a slot 75 in bottom 77 of cartridge 7, wherein at least one spring-loaded standby U-shaped staple 41 is located, the staple being disposed in this slot 75 substantially along the longitudinal axis of cartridge 7, with free pointed ends 79 facing the means for shaping—curvilinear surface 71. At least one standby U-shaped staple 41 is located in slot 75 of bottom 77 of cartridge 7 substantially under its flexible pusher 55 and is capable of extension from this slot 75 by action of spring 81 and at partial removal of flexible pusher 55 from the clearance between bottom 77 of cartridge 7 and its sliding lid 53 (FIG. 13-14). All cartridges 7 together with their sliding lids 53, flexible pushers 55 and bearing levers 49 are identical and interchangeable.

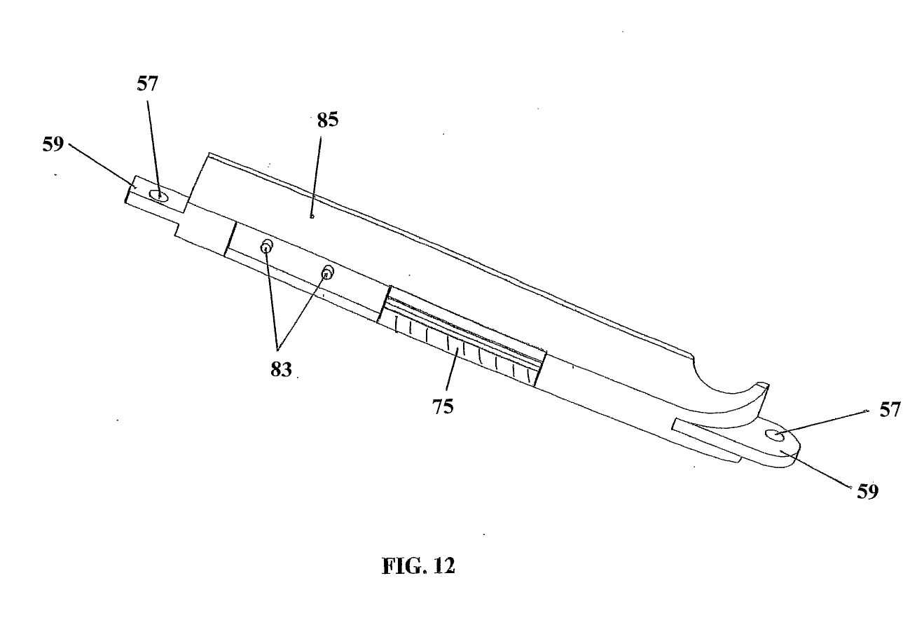

FIG. 12-14 show an embodiment of cartridge 7, having a through slot 75 in its bottom 77, with standby U-shaped staple 41 located in this slot 75 immediately under flexible pusher 55 and loaded on the opposite side by spring 81 secured in the recess of bottom 77 of cartridge 7 via specially provided bulges 83 (FIG. 12).

Each cartridge 7 of endovascular apparatus 1 contains at least one basic and at least one standby fastener means, substantially U-shaped staples, 39 and 41 respectively. All these staples 39 and 41 are fabricated from one of the materials of a group including stainless steel, titanium and shape memory alloys.

All cartridges 7 of proposed apparatus 1 are shaped substantially as a polyhedral prism (FIG. 11, 12) with two skewed side faces 85 inclined at an acute angle to one another. The vertex of this acute angle is located on the longituinal axis of expandable working head 5. All cartridges 7 are provided with means for connection with this expandable working head containing arms 59 with holes 57 which project from both bases of a corresponding polyhedral prism and are integral therewith.

Cartridges 7 have a body with a bottom 77 provided with a recess 87 (FIG. 11) for location of at least one basic fastener means, substantially U-shaped staple 39, and through slot 75 for location of at least one standby fastener means, substantially standby spring-loaded U-shaped staple 41. Further, the body of each cartridge 7 has slots 91 (FIG. 11) for mounting sliding lid 53, as well as a longitudinal duct 93 (FIG. 13) for location of at least one flexible pusher 55. Basic U-shaped staple 39 is located in its recess 87 in frictional contact with sliding lid 53, which prevents it from falling out of this recess 87. Standby spring-loaded U-shaped staple 41 is located in its through slot 75 in frictional contact with flexible pusher 55, which prevents it from extension from this through slot 75 before partial removal of flexible pusher 55 (FIG. 13-14). Recess 87 in the body of cartridge 7 is symmetric about the longitudinal axis of this cartridge 7 and parallel with its outer surface 95 (FIG. 11, 14). The means for shaping 71 of U-shaped staple 39 is shaped as a curvilinear guiding surface 71 which connects the bottom of recess 87 for U-shaped staple 39 with outer surface 95 of cartridge 7. This curvilinear guiding surface 71 has shaping grooves 97 diverging at an acute angle to one another and to the longitudinal axis of cartridge 7 (FIG. 11, 14).

Each U-shaped staple 39 or 41 is located in recess 87 or slot 75 of the body of a corresponding cartridge 7 in such a way that its pointed ends 69 and 79 face corresponding shaping grooves 97 on curvilinear guiding surface 71 of cartridge 7. This provides helical oppositely directed curling of pointed ends 69 or 79 of a corresponding U-shaped staple 39 or 41 during its movement over recess 87 of the body and shaping grooves 97 of curvilinear guiding surface 71 of cartridge 7 (FIGS. 11, 13, 14).

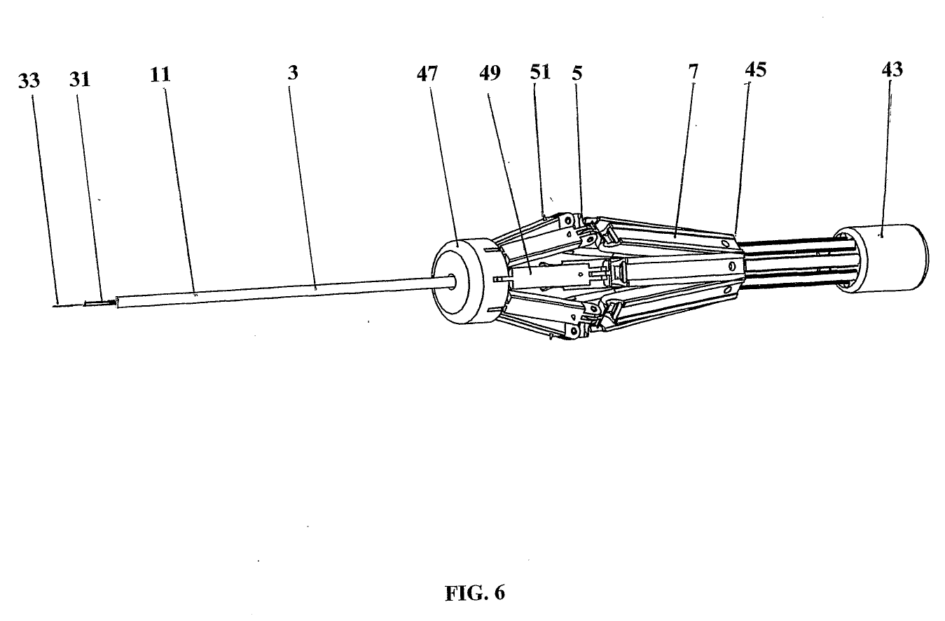

Apparatus 1 is additionally provided with means for axial and radial positioning at a given point of the operated blood vessel. The means for axial positioning contains a measuring scale on the surface of tubular body 3. The means for radial precision positioning of working head 5 contains markers—X-ray contrast marks evenly applied on the outer surface of cartridges 7 near the points of their connection with bearing levers 49 and serving to orient expandable working head 5 by its operation angle (FIGS. 5, 6).

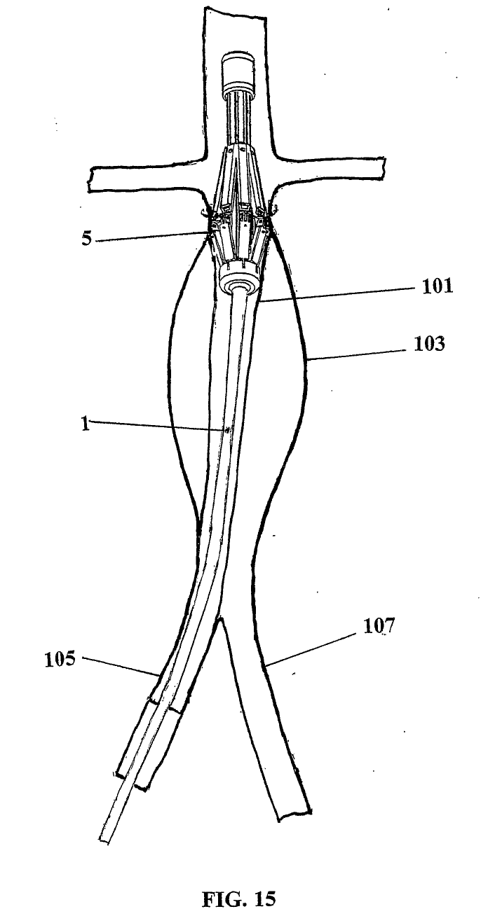

Another subject matter of the present invention is a method for delivery and fixation of a graft or stent-graft to the wall of a blood vessel from inside the latter. This method comprises several successive steps. At the first step graft or stent-graft 101 is prepared for delivery, mounted and secured on the surface of expandable working head 5 of apparatus 1, as well as crimped to an assigned outer diameter (FIG. 15).

At the second step endovascular apparatus 1 is brought into operative position, inserted into a corresponding blood vessel, in this case aorta 103 (FIG. 15), expandable working head 5 is brought to the securing area, and precision axial and radial positioning of apparatus 1 is accomplished at a given point of aorta 103 via a measuring scale on the surface of tubular body 3 and via markers—X-ray contrast marks on the surface of cartridges 7. Then working head 5 is deployed in such a way that the delivered graft or stent-graft 101 comes in contact with the inner surface of aorta 103 at the moment of their mutual fixation, and cartridges 7 of this expandable working head 5 are located near corresponding suturing points (FIG. 15). All this is performed by a surgeon under X-ray control.

Expandable working head 5 is deployed as follows (FIG. 15). When this head 5 reaches the given securing point, the surgeon rotates pivoting head 25 of control mechanism 9. This sets first bearing bush 45 in motion towards second bearing bush 47, whereby cartridges 7 and bearing leavers 49 turn in their pivotal connections and extend in radial direction causing the expansion of working head 5. This, in its turn, enables the prosthesis-graft or stent-graft 101 delivered by appartus 1 to be stretched near its end by expandable working head 5, pressed by its edge against the inner surface of a corresponding blood vessel, in this case aorta 103, and additionally stretch to a certain extent together with aorta 103, so as to entirely stretch the aorta in the area of its future connection with this prosthesis 101 (FIG. 15). The surgeon controls therewith the extent of stretching of working head 5 via visual information obtained from an X-ray monitor (not shown in the drawings). When necessary, stretching of aorta 103 and graft or stent-graft 101 may be slightly reduced by rotating pivoting head 25 in reverse direction.

Then U-shaped staples 39 are set in motion, which is accomplished by action of flexible pushers 55 activated, in their turn, via pressure bush 43, second connecting means—tie 33 and second control means—pressure handle 27 which are associated with these pushers. As a result, pointed ends 69 of each of the U-shaped staples 39 enter shaping grooves 97 of curvilinear guiding surface 71 of a correspnding detachable cartridge 7, are curled therein, diverging in opposite directions from one another, pierce the wall of graft or stent-graft 101 and the surrounding wall of aorta 103 and return again over a spiral to curvilinear guiding surface 71, repeating this rotation if permitted by the given length of staples 39. As a result, the wall of graft or stent-graft 101 gets sutured by wire spirals formed from U-shaped staples 39 to a corresponding portion of the wall of aorta 103, and U-shaped staples 39 themselves entirely extend from recesses 87 of corresponding cartridges 7 and are released from these cartridges 7, forming an oversew suture at the end of a corresponding graft or stent-graft 101 (FIG. 15).

Then the cartridges are recharged with standby U-shaped staples 41. This is accomplished by withdrawing swing lock 35 which enables pressure handle 27 to turn through a certain angle by action of return spring 37. All this causes a partial withdrawal of flexible pushers 55 which, in their turn, are activated via pressure bush 43, second connecting means—tie 33 and second control means—pressure handle 27 associated with these pushers. As a result, each of the spring-loaded standby U-shaped staples 41 extends from through slot 75 of a corresponding cartridge 7, and then, at reverse motion of a corresponding flexible pusher 55, moves by action of the latter into recess 87 wherein basic U-shaped staple 39 was located before. As a consequence, endovascular apparatus 1 is made ready for repeated suturing of graft or stent-graft 101 (FIG. 15).

When repeated suturing is necessary, standby U-shaped staples 41 are set in motion. This is accomplished due to flexible pushers 55, which are, in their turn, activated via pressure bush 43, second connecting means—tie 33 and second control means—pressure handle 27 associated with these flexible pushers much as basic staples 39 were extended before. As a result, pointed ends of each of the standby U-shaped staples 41 enter shaping grooves 97 of curvilinear guiding surface 71 of a corresponding detachable cartridge 7, are curled therein,. diverging in opposite directions from one another, pierce the wall of graft or stent-graft 101 and the surrounding wall of aorta 103 and return again over a spiral to curvilinear guiding surface 71, repeating this rotation if permitted by the given length of staples 41. In consequence, the wall of graft or stent-graft 101 gets repeatedly sutured by wire spirals formed from standby U-shaped staples 41 to a corresponding wall portion of aorta 103, and standby U-shaped staples 41 themselves entirely extend from recesses 87 of corresponding cartridges 7 and are released from these cartridges 7, forming a second oversew suture at the end of a corresponding graft or stent-graft 101 (FIG. 15).

A second oversew suture may be formed at the same end of graft or stent-graft 101 as the first one. This may be accomplished by some radial turning or axial displacement of working head 5 relative to aorta 103. It is evident that to do so it is necessary to completely contract working head 5, turn and displace it, deploy again and orient this working head 5 relative to aorta 103, and only then form the second oversew suture. The second oversew suture may be formed via standby U-shaped staples 41 also at the second end of graft or stent-graft 101 for its securing from the other side in aorta 103 itself or in one of bifurcations 105 or 107 (FIG. 15).

At last, at the final step, sutured graft or stent-graft 101 is detached from apparatus 1. This is accomplished by turning the pivoting head 25 which enables working head 5 to return to its initial position, and radial tongues 51 extend from graft or stent-graft 101 and are detached therefrom. Then endovascular apparatus 1 is brought into inoperative position and removed from the prosthesis—graft or stent-graft 101 and from aorta 103 (FIG. 15).

The application of proposed apparatus 1, as well as the realization, on the basis of this apparatus, of a new and improved method for securing prostheses-grafts or stent-grafts 101 to the wall of a blood vessel, substantially aorta 103, allows to solve the problem of preventing grafts or stent-grafts 101 from displacement from a required position inside aorta 103 which may occur due to peristelsic oscillations of aorta walls or shakings of a patient's body during his movement.

While this invention has been described in conjunction with specific embodiment thereof, it is evident that many alternatives, modifications and variations will be apparent to those skilled in the art. Accordingly, the preferred embodiments of the invention as set forth herein are intended to be illustrative, not limiting. Various changes may be made without departing from the spirit and scope of the invention as defined in the following claims.

1-31. (canceled)

32. Device for delivery and oversew fixation of endovascular grafts or stent-grafts, said device comprising:
a tubular body configured for positioning within a body lumen into which a graft or stent-graft has been previously disposed, said tubular body being rigid in a longitudinal direction and flexible in a lateral direction;
an expandable working head disposed near a first end of said tubular body, said expandable working head comprising a plurality of cartridges each retaining therein a fastener and being adapted to impart progressive motion to the fasteners and to shape them during their extension from the cartridges; and
a control mechanism located at a second end of said tubular body opposite to the working head, and operatively coupled to the working head through the tubular body, said control mechanism having a first actuator for controlling expansion and contraction thereof, and a second actuator serving to control the extension of a respective fastener from each of the cartridges, as well as to control recharging of each of said cartridges with at least one standby fastener stored in the cartridge, whereby linear motion of the tubular body within the graft or stent-graft combines with rotation of the working head to release successive fasteners and fix the graft or stent-graft to an internal wall of said body lumen.

33. The device according to claim 32, wherein said expandable working head contains a pressure bush and first and second bearing bushes arranged in succession one after another beginning from the free end of this working head and in alignment with said tubular body, as well as an axial tube rigidly connected with the first bearing bush and passing via a through axial hole in the second bearing bush.

34. The device according to claim 32, wherein said expandable working head is provided at least with eight cartridges pivotally mounted at one end to a first bearing bush and pivotally connected at opposite ends to bearing levers which are, in turn, pivotally mounted on a second bearing bush.

35. The device according to claim 34, wherein said bearing levers are provided with means for securing a delivered graft or stent-graft shaped substantially as radial tongues disposed at one of the ends of the bearing levers, near their pivotal connections with corresponding cartridges.

36. The device according to claim 32, wherein each of said cartridges contains a shaping unit for shaping the fasteners, each shaping unit being disposed in the respective cartridge for shaping the fastener as it is withdrawn from the cartridge.

37. The device according to claim 36, wherein each of said cartridges includes means for retaining at least one basic fastener in the cartridge.

38. The device according to claim 37, wherein each of said cartridges includes a pusher for imparting motion to the fasteners, said pusher being rigid in a longitudinal direction and flexible in a lateral direction.

39. The device according to claim 36, wherein each of said cartridges has a lower surface and a sliding lid and contains a storage means for storing at least one standby fastener, said storage means having a slot in the lower surface of the cartridge wherein there is disposed along a substantially longitudinal axis of the cartridge at least one spring-loaded standby U-shaped staple having free pointed ends facing the shaping unit, said at least one standby staple being disposed under the flexible pusher and being capable of extension from the slot by action of the spring and partial removal of the flexible pusher from a clearance between the lower surface of the cartridge and the sliding lid.

40. The device according claim 32, wherein each basic fastener and each standby fastener is formed of a substantially U-shaped stapled fabricated from a material in the group including stainless steel, titanium and shape memory alloys.

41. The device according to claim 32, wherein said expandable working head is provided with at least eight cartridges evenly and circumferentially arranged around a longitudinal axis of the working head, each cartridge being pivotally connected at one end to a first bearing bush and being pivotally connected at an opposite end to bearing levers which are pivotally mounted on a second bearing bush.

42. The device according to claim 32, further including a measuring scale on the surface of said tubular body for axial precision positioning thereof.

43. The device according to claim 32, wherein the first actuator comprises:
a hollow body having a first end and a second end and being rigidly connected at its first end to said working head via the tubular body, and
a slider adapted to reciprocate within said hollow body and being having a retaining handle rigidly attached thereto, operatively associated with a pivoting head located at the second end of the hollow body.

44. The device according to claim 43, wherein the second actuator comprises:
a pressure handle pivotally attached to the retaining handle and having a short free end enclosed in the slider, the slider being rigidly connected to a first connecting device, and the short free end of said pressure handle being operatively associated with a second connecting device.

45. The device according to claim 44, wherein said pressure handle is supplied with a swing lock pivotally mounted on said slider, as well as a flat return spring attached by one its end to this pressure handle, and by its second end to said retaining handle.

46. The device according to claim 39, wherein lower surface of the cartridge is provided with a recess for location of at least one substantially U-shaped basic staple, and a through slot for location of at least one spring-loaded substantially U-shaped standby staple, the basic staple being disposed in said recess in frictional contact with the sliding lid, and the standby staple being disposed in the through slot in frictional contact with the flexible pusher.

47. The device according to claim 46, wherein the recess is symmetrical about a longitudinal axis of the cartridge and parallel with an outer surface, the cartridge being provided with a shaping means for shaping the staple during its extension from the recess.

48. The device according to claim 47, wherein the shaping means includes a curvilinear guiding surface connecting the bottom of the recess with the outer surface of the cartridge, said curvilinear guiding surface having shaping grooves.

49. The device according to claim 48, wherein said shaping grooves diverge at an acute angle from one another and a longitudinal axis of the cartridge.

50. The device according to claim 48, wherein each of the U-shaped staples has opposing pointed free ends and is disposed in said recess such that its pointed ends face corresponding shaping grooves on said curvilinear guiding surface.

51. A method for using the device of claim 32 for delivery and fixation of a graft or stent-graft to an inside wall of a blood vessel, the method comprising:
i) preparing the graft or stent-graft for delivery, its mounting and securing on the surface of said expandable working head of the device for its delivery and fixation, as well as its crimping to an assigned outer diameter;
ii) introducing the device in an operative position into a corresponding blood vessel, bringing said expandable working head to the securing area, effecting precision axial and radial positioning of the device at a given point of the blood vessel deploying the working head so that the delivered graft or stent-graft is in contact with an inner surface of the blood vessel at the moment of their mutual fixation, and the cartridges of the expandable working head are located near corresponding suturing points;
iii) pushing the U-shaped staples so that pointed ends of each of U-shaped staples enter shaping grooves of a curvilinear guiding surface of a corresponding detachable cartridge, are curled therein, diverging in opposite directions to one another, pierce the graft or stent-graft wall and the surrounding blood vessel wall and return to said curvilinear guiding surface, so as to suture the graft or stent-graft wall by wire spirals formed from U-shaped staples to a corresponding portion of the blood vessel wall, said U-shaped staples entirely extending from recesses of corresponding cartridges and being released from said cartridges, forming an oversew suture at the end of a corresponding graft or stent-graft;
iv) recharging the cartridges with standby U-shaped staples by partial removal of said flexible pushers, whereby each of the standby staples extends from the through slot of a corresponding cartridge, and then, on reverse motion of a corresponding flexible pusher, moves into said recess, so as to ready the device for repeated suturing;
v) repeating suturing, if necessary using the standby staples so as to form a second oversew suture at the end of a corresponding graft or stent-graft;
vi) detaching the delivered and sutured graft or stent-graft from the device for delivery and fixation, and
vii) bringing the device for delivery and fixation into inoperative position and its removal from the graft or stent-graft and from the blood vessel.
